(19) United States
(12) Patent Application Publication
(10) Pub. No.: US 2026/0083493 A1
(43) Pub. Date: Mar. 26, 2026

(54) ELECTROSURGICAL DEVICE AND METHOD OF USE

(71) Applicant: Meditrina, Inc., San Jose (CA)

(72) Inventors: Csaba TRUCKAI, San Jose, CA (US); John H. SHADDUCK, San Jose, CA (US)

(73) Assignee: Meditrina, Inc., San Jose, CA (US)

(21) Appl. No.: 19/331,558

(22) Filed: Sep. 17, 2025

Related U.S. Application Data
(60) Provisional application No. 63/698,413, filed on Sep. 24, 2024.

(51) Int. Cl. A61B 18/14; A61B 18/00; A61B 18/12

(52) U.S. Cl. CPC A61B 18/14; A61B 18/1206; A61B 2018/00077; A61B 2018/00083; A61B 2018/00166; A61B 2018/00577; A61B 2018/0091; A61B 2018/00982; A61B 2018/126; A61B 2018/1472; A61B 2218/007

(57) ABSTRACT

Bipolar electrosurgical devices and methods of use for cutting, ablating, or coagulating tissue, for example, in endoscopic gynecology procedures.

### RELATED APPLICATION INFORMATION

The application is a non-provisional application of U.S. Provisional application No. 63/698,413 filed Sep. 24, 2024. The entirety of which is incorporated by reference.

### FIELD OF THE INVENTION

The present invention relates to bipolar electrosurgical devices and methods of use for cutting, ablating, or coagulating tissue, for example, in endoscopic gynecology procedures.

### SUMMARY OF THE INVENTION

The present disclosure relates to bipolar electrosurgical devices and methods of use. For example, a electrosurgical device can comprise a handle and a small diameter, elongated shaft adapted for introduction through a working channel of an endoscope. The RF device can include a mechanism for providing a high-pressure flow of saline through a small diameter jetting channel in the working end and a bipolar electrode arrangement adapted to create an RF plasma for ablating or coagulating targeted tissue in a saline-filled working space.

In another variation, an electrosurgical device can include a probe having a handle coupled to an elongate shaft with a working end; an RF source operatively coupled to a controller; a source of a conductive fluid communicating with a jetting channel having a jetting outlet in an electrically insulative component of the working end; a pump mechanism operatively coupled to the controller configured to provide a conductive fluid flow at a flow rate from 5 ml/min to 25 ml/min in the jetting channel; a first polarity electrode exposed to the conductive fluid flow proximal to the jetting outlet; a second polarity electrode at the working end spaced apart from the jetting outlet; and wherein the RF source and controller are adapted to couple RF current to the conductive fluid flow in the jetting channel to generate a plasma at the jetting outlet.

The jetting channel can have a mean cross-section ranging from 0.05 mm to 0.5 mm and/or a length ranging from 1 mm to 10 mm. Additionally, the jetting channel can have a mean cross section ranging from 0.05 mm to 0.5 mm

The devices described herein can further include a negative pressure source operatively coupled to the controller, the negative pressure source communicating with an aspiration channel in the elongate shaft. The aspiration channel can extend to an aspiration window in the working end.

In additional variations, the negative pressure source is operatively coupled to the controller.

Variations of the device include a source of a conductive fluid that is carried in the handle. Alternatively, or in combination, the source of a conductive fluid is remote from the handle.

The pump mechanism can be carried in the handle or can be remote from the handle.

In additional variations, the pump mechanism comprises a first pump remote from the handle and a second booster pump in the handle.

Variations of the device can include an elongate shaft that is configured for articulation. Alternatively, or in combination, the elongate shaft can be detachable from an aspiration sleeve.

### BRIEF DESCRIPTION OF THE FIGURES

FIG. 1 is a perspective view of an RF device corresponding to the invention that has a handle connected to an elongate introducer assembly with a working end including a bi-polar electrode arrangement for igniting a plasma in a conductive fluid flow within a small diameter jetting channel together with a block diagram of an RF source, negative pressure source, fluid source, and controller operatively coupled to the device.

FIG. 2 an enlarged perspective view of the working end of the RF device of FIG. 1.

FIG. 3 is a sectional view of the working end of the RF device of FIGS. 1 and 2 illustrating a step in a method of igniting a plasma in a saline-filled working space.

FIG. 4 is a perspective view of another variation of a working end with a plasma jetting channel within a side window of the working end.

FIG. 5 is a sectional view of the working end of the RF device of FIG. 4.

FIG. 6A is a perspective view of another variation of an RF device with a shaft carrying the plasma jetting channel that can be de-coupled from an outer sleeve carrying the aspiration channel.

FIG. 6B illustrates the shaft carrying the plasma jetting channel of FIG. 6A in an assembled position with the outer sleeve carrying the aspiration channel of FIG. 6A.

FIG. 7 illustrates a step in a method of using the RF device of FIG. 6B in ablating targeted tissue in a patient's uterine cavity.

FIG. 8 is a sectional view of another variation of an RF device shaft carrying a plasma jetting channel and bi-polar electrode arrangement that can be used separately to ablate tissue or that can be assembled with an outer sleeve carrying an aspiration channel as in the variation of FIGS. 6A-6B.

FIG. 9 is a perspective view of another variation of an RF device with a working end comprising a ceramic or other dielectric housing that carries both the plasma jetting channel and a window coupled to the negative pressure source.

### DETAILED DESCRIPTION

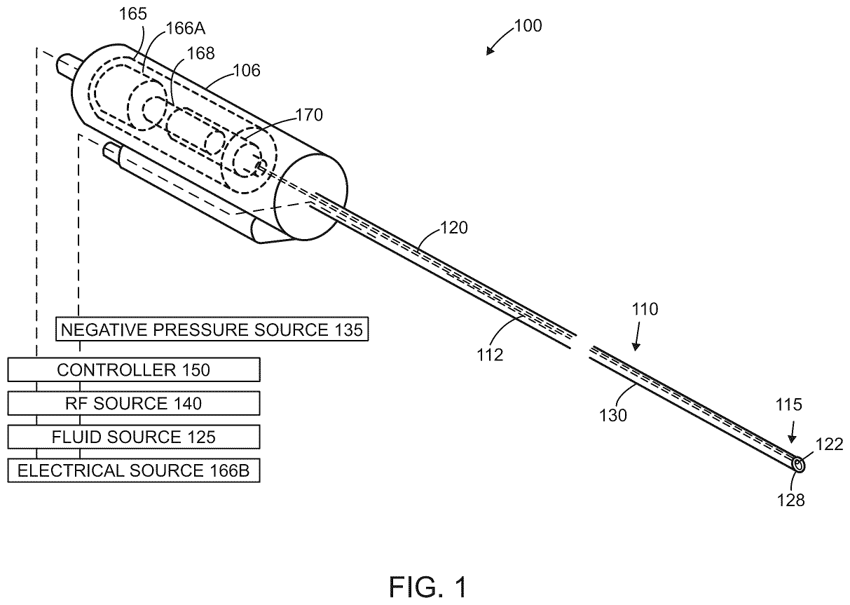

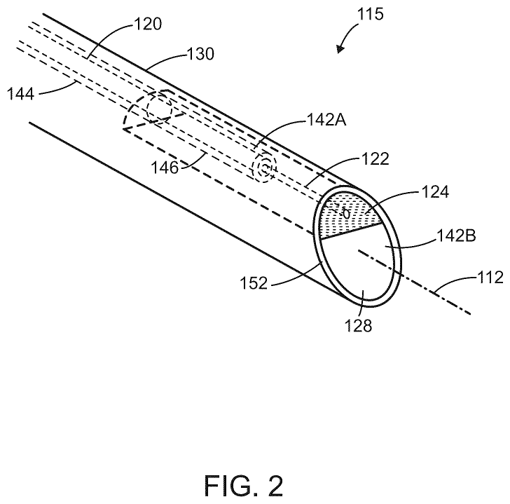

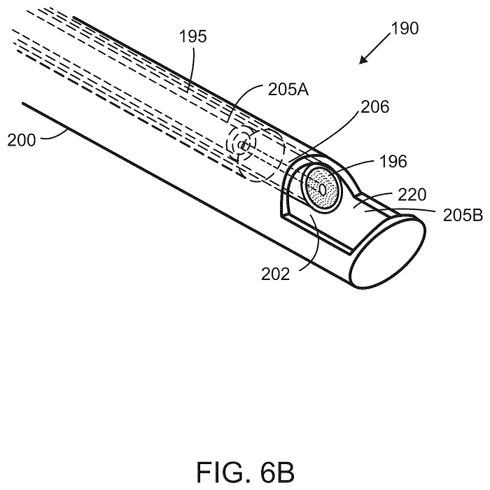

FIGS. 1 and 2 illustrate an electrosurgical (RF) device 100 comprising a handle 106 coupled to an elongated introducer shaft 110 extending about a longitudinal axis 112 and having a diameter typically ranging from 3 mm to 8 mm. The length of the introducer shaft 110 is highly elongated and suited for introduction through the working channel of an endoscope 114 (see FIG. 7). As will be described below, the RF device 100 is adapted for use in a fluid-filled working space in a patient's body, such as a uterine cavity filled with a conductive saline fluid.

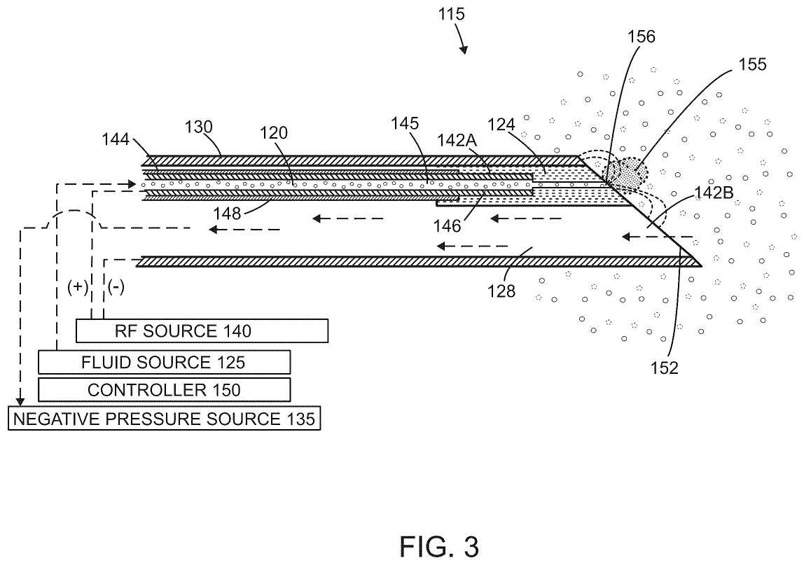

In the variation of FIG. 1, the introducer shaft 110 has a working end 115, as shown in the enlarged views of FIGS. 2 and 3. The shaft 110 and working end 115 have an inflow channel 120 therein that transitions to a small diameter jetting channel 122 in an insulative component 124 in the working end 115. A remote fluid source 125 is in fluid communication with the inflow channel 120 and jetting channel 122. In FIGS. 2 and 3, it can be seen that the working end 115 further is configured with a larger cross-section outflow or aspiration channel 128 in a thin-wall outer sleeve 130 of the shaft 110 that communicates with a negative pressure source 135.

As best seen in the sectional view of FIG. 3, a remote RF source 140 is coupled to first and second polarity electrodes 142A and 142B in the working end 115. In the variation of FIGS. 1-3, the inflow channel 120 consists of the lumen of an electrically conductive hypotube 144 which comprises the first polarity electrode 142A. The hypotube 144 functions as the first polarity electrode 142A to couple RF current to a flow of saline 145 (FIG. 3) in the inflow channel 120. In FIG. 3, it can be seen that the distal end 146 of the hypotube 144 is embedded in the insulative component 124 which comprises a ceramic or other dielectric material. The portion of the hypotube 144 proximal to the insulative component 124 and exposed to the aspiration channel 128 is covered with an electrically insulative coating 148, such as heat-shrink polymer (FIG. 3).

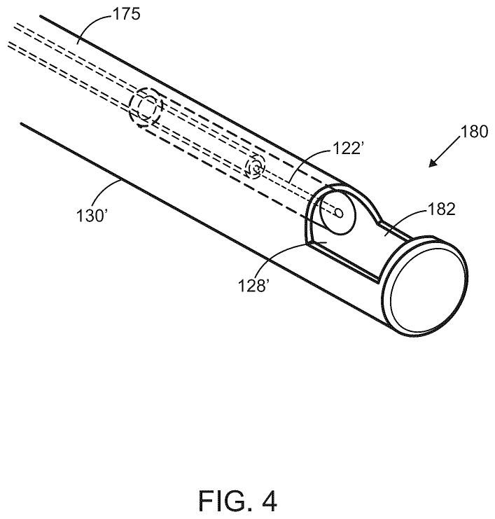

Now referring to FIG. 3, the thin-wall outer sleeve 130 consists of a conductive material, such as a stainless steel tube, that comprises the second polarity electrode 142B. The RF source 140 and a controller 150 are operatively coupled to the first and second polarity electrodes 142A and 142B. The controller 150 is configured to control the operating parameters of the RF source 140, the negative pressure source 135, and the fluid source 125. In many endoscopes, the working channel is approximately 4.0 mm in diameter; thus, the outer sleeve 130 often has an outer diameter D ranging from 3.5 mm to 3.95 mm. In the variation of FIG. 1 to 3, the distal surface 152 of the working end 115 is angled, but it should be appreciated that the distal surface 152 can take a variety of shapes, including any angled shape, a surface that is 90°to the longitudinal axis 112, and other shapes with a window as described below in the variations of FIGS. 4 to 6B below.

One objective of the device shown in FIGS. 1 to 3 is to create or ignite an RF plasma 155 at the distal outlet 156 of the jetting channel 122, which can be understood from FIG. 3. As can be seen in the sectional view of FIG. 3, the first polarity electrode 142A is proximal to the dielectric member 124 carrying the jetting channel 122 and the second polarity electrode 142B comprises a surface of the outer sleeve 130 which is distal from jetting channel 122. Thus, delivering RF current to the electrodes 142A 142B couples energy to the conductive saline 145 in the jetting channel 122, which ignites an RF plasma therein, or at the distal outlet 156 of the jetting channel 122. The diameter and length of the jetting channel 122 thus causes very high energy densities therein, which can instantly ignite a plasma in the saline 145 flowing through the jetting channel 122. It can be easily understood that the parameters of the saline inflows and RF delivery are key factors in igniting the plasma instantly in the channel 122 or the distal outlet 156 thereof.

In a variation, the diameter of the jetting channel 122 is small and can range from 0.05 mm to 0.5 mm. The length of the jetting channel 122 can range from 1 mm to 10 mm. The flow rate of saline 145 through the jetting channel 122 can range from 5 ml/min to 25 ml/min then results in a PSI ranging between 500 PSI and 1,500 PSI in the jetting channel 122.

In a variation, the fluid source 125 is remote and includes a pump (not shown) that delivers saline 145 to the handle 106 of the RF device 100 at a relatively low pressure, for example, at less than 250 PSI in the variation of FIG. 1. In this variation, a booster pump 165 is provided in the handle 106 that consists of a syringe pump as is known in that art and comprises an electric motor 166A coupled to electrical source 166B driving a plunger 168 in a syringe 170. The booster pump 165 then increases the fluid pressure in the flow channel and jetting channel to the range of 500 PSI to 1,500 PSI, as described above.

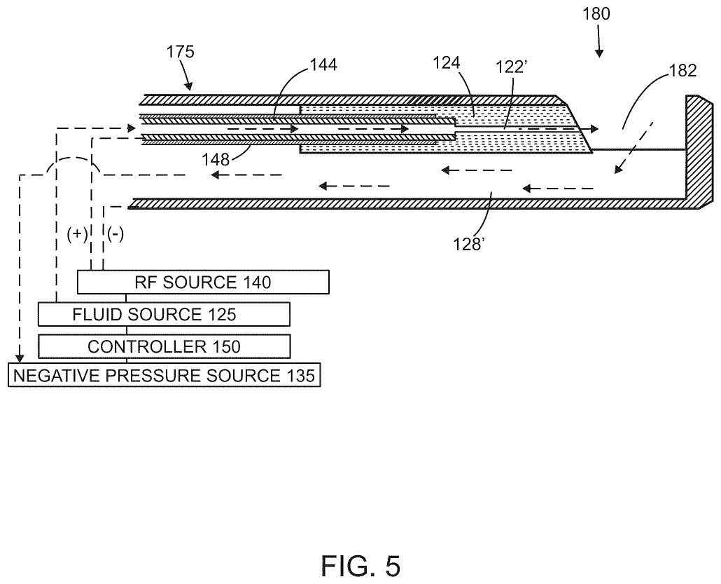

Now turning to FIGS. 4 and 5, another variation of an RF device with a shaft 175 and working end 180 is shown that is similar to the variation of FIGS. 1 to 3 except that the outer sleeve 130′ forms the working end 180 with a window 182 distal to the jetting channel 122′. In all other aspects, the working end 180 of FIGS. 4 and 5 operates as described previously. The window 182 can be pressed against targeted tissue as the plasma 155 (FIG. 3) ablates tissue wherein the negative pressure source 135 extracts tissue debris through the window 182 and aspiration channel 128′.

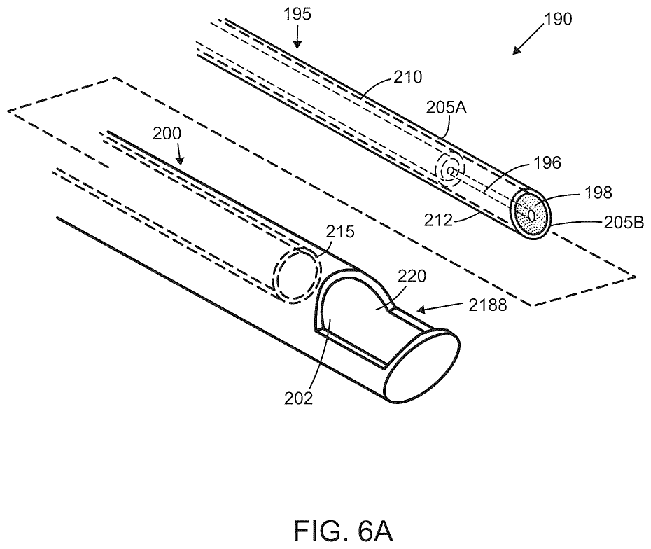

Now, turning to FIGS. 6A and 6B, another variation of a working end 190 is shown that is similar to that of FIGS. 4 and 5 except the shaft 195 carrying the jetting channel 196 in dielectric member 198 is de-detachable from an aspiration sleeve 200 carrying the aspiration channel 202. In this variation, the first polarity electrode 205A comprises an interior hypotube 210, as described previously. The second polarity electrode 205B comprises an outer sleeve 212 of the shaft 195. This configuration allows the aspiration sleeve 200 to consist of a low-cost extruded polymer material that carries a lumen 215 for receiving the shaft 195. The distal end 218 of the aspiration sleeve 200 is modified to provide a window 206, as shown in FIG. 6B. In all other aspects, the variation of FIGS. 6A-6B functions as described above.

In the variation of FIGS. 6A-6B, the outer aspiration sleeve 200 comprises a non-conductive polymer, which is important so that RF current is not coupled to the endoscope shaft. In other variations, the outer surface of the shaft of the RF device is covered with a non-conductive layer to ensure that RF current is not coupled to the endoscope shaft. The second polarity electrode comprises an exposed surface of the outer sleeve inside the window.

Now, turning to FIG. 7, a method of the invention is shown in an exemplary procedure of introducing an RF device with the working end 180 of FIGS. 4 and 5 into a patient's uterine cavity 220 to ablate targeted tissue T. FIG. 7 shows an endoscope 114 being introduced into the patient's uterine cavity 220 with saline 145 introduced through the endoscope 114 to distend the uterine cavity as is known in the art. The endoscope is of the type disclosed in U.S. Pat. No. 11,998,172. A tenaculum 222 is used to grip the cervix 224 as is known in the art, and a cervical seal 226 is pressed against the cervix to prevent outflow of the saline distension fluid. The shaft 175 of the RF device is introduced through the working channel WC of endoscope 114, wherein the field of view of the endoscope's image sensor allows for viewing targeted tissue T. The system is activated by the clinician to ignite a plasma in the jetting channel (FIG. 5) to ablate target tissue T, while the negative pressure source 135 extracts ablated tissue debris through the aspiration channel 128′ (FIG. 5).

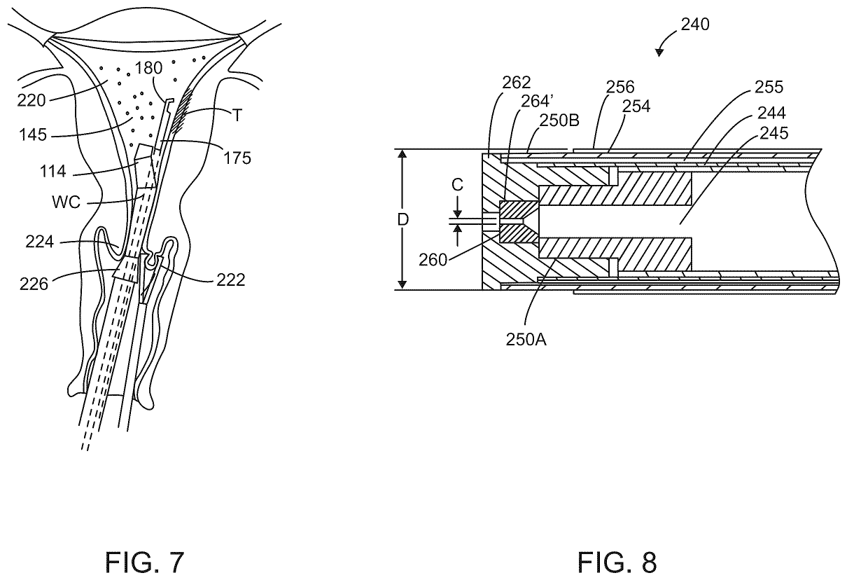

FIG. 8 is a sectional view of another variation of working end 240 that is similar to that of FIG. 6A which shows additional components for enabling the ignition of an RF plasma. In the working end 240 of FIG. 8, the inner sleeve 244 terminates distally in a conductive block 245 that comprises the first polarity electrode 250A. An outer sleeve 254 spaced apart from the inner sleeve 244 by an insulator layer 255, wherein the outer sleeve 254 with diameter D ranging from 1 mm to 3 mm comprises the second polarity electrode 250B with an insulative layer 256 covering all but a distal portion of the outer sleeve 254. The jetting channel 260 with diameter C is within a dielectric assembly that includes a ceramic component 262 together with a sapphire, gem, or glass-like component 264 that potentially is stable over time to resist degradation by the ignition of the plasma therein. In all other aspects, the working end 240 functions as described previously.

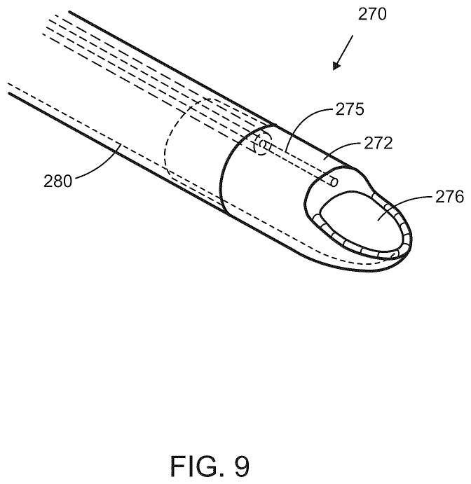

FIG. 9 is a perspective view of another variation of a working end 270 wherein a distal component 272 of the working end comprises a dielectric material, such as a ceramic, that houses both the jetting channel 275 and an aspiration window 276 and aspiration channel 280. In all other aspects, this variation functions as described previously.

In another variation (not shown), the working end can comprise an articulating or deflecting section that comprises an assembly of co-axial thin-wall slotted sleeves with an outer slotted sleeve and an inner slotted sleeve as are known in the art, for example, as shown in U.S. Pat. No. 10,058,336 by Truckai et al. issued Aug. 28, 2018.

The RF device, as described above, can be used in multiple procedures in gynecology, urology, arthroscopy, and the like in a saline-filled working space. Although particular variations of the present invention have been described above in detail, it will be understood that this description is merely for purposes of illustration, and the above description of the invention is not exhaustive. The variations above are shown with a moto drive for moving a cutting sleeve helically and rotationally, but manually operated mechanisms are also possible for either or both such helical and rotational movements. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item includes the possibility that there are plural of the same items present. More specifically, as used herein and in the appended claims, the singular forms “a,” “and,” “said,” and “the” include plural references unless the context clearly dictates otherwise.

It is important to note that where possible, aspects of the various described embodiments or the embodiments themselves can be combined. Where such combinations are intended to be within the scope of this disclosure.

What is claimed is:

1. An electrosurgical device for ablating tissue, comprising:
a probe having a handle coupled to an elongate shaft with a working end;
an RF source operatively coupled to a controller;
a source of a conductive fluid communicating with a jetting channel having a jetting outlet in an electrically insulative component of the working end;
a pump mechanism operatively coupled to the controller configured to provide a conductive fluid flow at a flow rate from 5 ml/min to 25 ml/min in the jetting channel;
a first polarity electrode exposed to the conductive fluid flow proximal to the jetting outlet;
a second polarity electrode at the working end spaced apart from the jetting outlet; and
wherein the RF source and controller are adapted to couple RF current to the conductive fluid flow in the jetting channel to generate a plasma at the jetting outlet.

2. The electrosurgical device of claim 1 wherein the jetting channel has a mean cross-section ranging from 0.05 mm to 0.5 mm.

3. The electrosurgical device of claim 1 wherein the jetting channel has a length ranging from 1 mm to 10 mm.

4. The electrosurgical device of claim 1 further comprising a negative pressure source operatively coupled to the controller, the negative pressure source communicating with an aspiration channel in the elongate shaft.

5. The electrosurgical device of claim 4 wherein the aspiration channel extends to an aspiration window in the working end.

6. The electrosurgical device of claim 4 wherein the negative pressure source is operatively coupled to the controller.

7. The electrosurgical device of claim 1 wherein the source of a conductive fluid is carried in the handle.

8. The electrosurgical device of claim 1 wherein the source of a conductive fluid is remote from the handle.

9. The electrosurgical device of claim 1 wherein the pump mechanism is carried in the handle.

10. The electrosurgical device of claim 1 wherein the pump mechanism is remote from the handle.

11. The electrosurgical device of claim 1 wherein the pump mechanism comprises a first pump remote from the handle and a second booster pump in the handle.

12. The electrosurgical device of claim 1 wherein the jetting channel has a mean cross section ranging from 0.05 mm to 0.5 mm.

13. The electrosurgical device of claim 1 wherein the elongate shaft is configured for articulation.

14. The electrosurgical device of claim 1 wherein the elongate shaft is detachable from an aspiration sleeve.
